(19) United States
(12) Patent Application Publication
(10) Pub. No.: US 2009/0304773 A1
(43) Pub. Date: Dec. 10, 2009

(54) SURGICAL ADHESIVE AND USES THEREFORE

(75) Inventors: Michael T. Milbocker, Holliston, MA (US); Dix P. Poppas, Larchmont, NY (US)

Correspondence Address: RISSMAN HENDRICKS & OLIVERIO, LLP, 100 Cambridge Street, Suite 2101, BOSTON, MA, 02114, US

(73) Assignee: Promethean Surgical Devices, LLC, Woburn, MA (US)

(21) Appl. No.: 12/542,203

(22) Filed: Aug. 17, 2009

Related U.S. Application Data
(63) Continuation of application No. 11/010,629, filed on Dec. 9, 2004.
(60) Provisional application No. 60/528,150, filed on Dec. 9, 2003.
(60) Provisional application No. 60/541,537, filed on Feb. 3, 2004.
(60) Provisional application No. 60/557,314, filed on Mar. 29, 2004.
(60) Provisional application No. 60/557,411, filed on Mar. 29, 2004.

(51) Int. Cl. A61F 2/00; A61K 31/785

(52) U.S. Cl. 424/423; 424/78.37

(57) ABSTRACT

The present invention provides a liquid polymer composition which can be implanted into a living mammal and which forms a solid hydrogel by in situ polymerization upon contact with body fluid and tissue. The composition also can be used as a coating on a medical device, or for the formation of a medical device. Formation of a solid implant or coating involves crosslinking of the adhesive with itself and with surrounding tissue. The liquid implant, by itself or in conjunction with various prostheses, can be used for many purposed, including fixation of the urethra for providing treatment for incontinence, and repair of herniations in the abdominal cavity, including rectocele, cystocele, enterocele, and inguinal hernia. The adhesive may be used to establish adhesion prevention during such repairs, in part by coating or being the material of a repair mesh.

This application claims the benefit of the priority of U.S. Provisional Patent Applications 60/528,150, filed Dec. 9, 2003; 60/541,537, filed Feb. 3, 2004; 60/557,314, filed Mar. 29, 2004; and 60/557,411, filed Mar. 29, 2004. Each of these applications is incorporated herein in its entirety by reference, where permitted.

### FIELD OF THE INVENTION

The present invention provides a liquid polymer composition which can be implanted into a living mammal and which forms a solid hydrogel by in situ polymerization upon contact with body fluid and tissue. The composition also can be used as a coating on a medical device, or for the formation of a medical device. Formation of a solid implant or coating involves crosslinking of the adhesive with itself and with surrounding tissue or devices. The liquid implant, by itself or in conjunction with various prostheses, can be used for many purposes, including fixation of the urethra for providing treatment for incontinence, and repair of aneurysms or herniations in the abdominal cavity, including rectocele, cystocele, enterocele, and inguinal hernia. The adhesive may be used to establish adhesion prevention during such repairs, optionally in part by coating or being the material of a repair mesh.

### BACKGROUND OF THE INVENTION

Various medical conditions require fixation or repair of internal organs. In many cases, especially when suturing is required, a superior solution can in principle be provided by use of a surgical adhesive. As an example, stress incontinence, the involuntary loss of urine due to a sudden rise in intra-abdominal pressure, can be alleviated by fixation of the urethra. Present practice (for example as described in U.S. Pat. Nos. 6,334,446; 6,042,534; 6,221,005; 6,077,216; or 6,110,101) in some cases involves the use of a polymeric “sling”, which is complex to install, and which can lead to erosion of the urethra over time.

As another example, in hernia repair, supporting meshes are frequently used, and are typically held in place with sutures or staples. The placement of these meshes can complicate the procedure, and the staples or sutures can cause pain during the period of tissue ingrowth into the repair mesh. Examples of such procedures are described in U.S. Pat. Nos. 6,197,036; 6,382,214; 6,502,578; 6,503,190; 6,669,654; and 5,571,117.

One aspect of the invention, as describe below, is the use of an effective surgical adhesive in the simplification of such procedures. Surgical adhesives are known, but despite the attraction of repair using surgical adhesives, few procedures actually use them. Several factors contribute to this low level of use. Some adhesives do not adhere well to wet tissue, such as cyanoacrylates. Others have poor mechanical strength, and many have overly rapid biodegradation for long-term repairs (e.g., U.S. Pat. No. 5,156,613, U.S. Pat. No. 4,804,691, U.S. Pat. No. 6,211,335, and U.S. Pat. No. 6,123,667). Other difficulties with proposed adhesives include excessive or uncontrolled swelling, as described in U.S. Pat. No. 6,265,016, and a lack of control over the formation of tissue adhesions, which are desirable in some procedures and undesirable in others.

### SUMMARY OF THE INVENTION

The invention comprises improved tissue adhesives, and methods for their use in certain surgical procedures. In one aspect of the present invention, liquid adhesive compositions are provided that are capable of bonding tissue while forming a solid in situ. The liquid adhesives comprise an isocyanate capped polyether polyol, and a low molecular weight polyisocyanate. Preferred adhesives further comprise polyhydroxyl compounds, such as water, or polyether-polyols, to control swelling and minimize adhesion promotion.

In other aspects, the materials used as adhesives can be pre-formed as medical devices. They can be used as filaments in strengthening applied adhesives, or in forming meshes for tissue support. The devices can be predictably degradable in situ or can resist degradation. The adhesives of the invention can be constructed to have porosity, either as formed, or gradually developing after implantation or in-situ polymerization. Porosity can be generated in vivo via selective degradation of a component, or by segregation of incompatible components, or by leaching of soluble materials entrapped in the polymerized adhesive. Porosity ex vivo can be via mechanical punch, laser cutting, and other means known in the art for perforating sheets of implantable material.

The adhesives and products made from them are biocompatible and have controlled strength. They are suitable for use in any medical procedure in which an adhesive or a polymeric or fibrous implant is used. For example, the adhesives can be used in immobilization of the urethra for treatment incontinence. Immobilization of a hollow organ, such as a urethra, is an important application for a non-swelling surgical material. There are a variety of ways of using the adhesive, and this the example of urethral treatment illustrates several of them. The use can be very simple, by direct adherence of the urethra to an adjacent tissue site. The use can be more like current practice, in which the urethra is immobilized by a sling, and the sling is glued in place by the adhesive. In other embodiments, the sling can be padded and made less erosive, by a coating of the adhesive material. The sling can even be formed of pre-cured adhesive.

Immobilization of other organs by the same or similar procedures is also possible. In another example, the invention is used to repair defects in the abdominal wall or in abdominal organs. This may be a simple application, such as manually pressing a herniated organ back to its proper place, and using the adhesive to seal the lesion left behind. The adhesive can also hold a mesh in place to give added strength. The adhesive can be formulated to develop a mesh-like structure, or in general, porosity, during or after application to a site to be repaired. Such porosity or mesh openings can enhance tissue growth into the implant region for long term strengthening. In more complicated repairs, a mesh and/or a resilient pad, either of which may be made of the adhesive, or coated with cured adhesive, is sandwiched between the tissues involved in the herniation. For example, a procedure is described for repairing a rectocele using the materials of the invention. Similar procedures can be used for repair of other herniations, or closure of unwanted openings, or anastomosis of tissues.

### BRIEF DESCRIPTION OF THE FIGURES

FIG. 1 shows the anatomy of an indirect inguinal hernia.

FIG. 2 shows the location of a rectocele, and

FIG. 3 shows the rectocele after repair.

### DETAILED DESCRIPTION OF THE INVENTION

Liquid in-situ polymerizing agents suitable in the present invention include both absorbable and non-absorbable polymers. Which type is needed depends on the procedure being used. For instance, in the case of polymeric fixation slings for urethral immobilization, absorbable polymers are preferred since they allow tissue to grow into the porous sling. In the case of direct mid-urethra fixation using an adhesive, a nonabsorbable polymer is preferred since the fixation point is essential to achieving continence. Nonabsorbable polyurethane polymer compositions suitable for the present invention are described in U.S. Pat. No. 6,296,607, and in application US 2003/0135238. Degradable polymer adhesive compositions are described in US 2004/0068078 or its equivalent WO 2004/021983. Meshes and methods for their application are described in US 2002/0049503. The teachings of each of these patents and applications, and of other publications referred to in this specification, are incorporated herein in their entirety, in jurisdictions where such incorporation is permitted.

### Adhesive Compositions

The present invention includes implantable pre-polyurethane compositions that form solids in the body, and that optionally contain links that are spontaneously hydrolysable under in vivo conditions after implantation (“degradable”, or equivalently herein, “biodegradable”). In other embodiments, the compositions are not specifically designed to be biodegradable.

One type of hydrolysable link used to make a polymeric material biodegradable is an ester link. These may be formed in a polyurethane precursor when the polyol of the pre-polymer has been esterified with a hydroxy carboxylic acid. Preferred hydroxy carboxylic acids are alpha-hydroxy acids such as glycolic and lactic acids. Other hydroxy carboxylic acids including caprolactic, hydroxy butyric, and hydroxy propionic acids can be used. The esterification process involves heating the acid under reflux with the polyol until the acid and hydroxyl groups form the desired ester links. The higher molecular weight acids are lower in reactivity and may require a catalyst, making them less desirable. Many other hydroxy acids are known, and there is a large literature available to the artisan in the preparation of biodegradable materials.

In addition, numerous other chemistries for making degradable bonds are known in the art. For example, U.S. Pat. No. 6,639,014 describes types of chemical linkages used to provide biodegradability. Useful hydrolyzable groups include polymers and oligomers of glycolide, lactide, epsilon-caprolactone, other hydroxy acids, and other biologically degradable polymers that yield materials that are non-toxic or present as normal metabolites in the body. Preferred poly(alpha-hydroxy acids) are poly(glycolic acid), poly(DL-lactic acid) and poly(L-lactic acid). Other useful materials include, without limitation, poly(amino acids), polycarbonates, poly(anhydrides), poly(orthoesters), poly(phosphazines) and poly(phosphoesters). Polylactones such as poly(epsilon-caprolactone), poly(delta-caprolactone), poly(delta-valerolactone) and poly(gamma-butyrolactone), for example, are also useful. The biodegradable regions may have a degree of polymerization ranging from one up to values that would yield a product that was not substantially water soluble. Thus, monomeric, dimeric, trimeric, oligomeric, and polymeric biodegradable regions may be used.

Biodegradable regions can be constructed from any polymers or monomers having or forming linkages susceptible to biodegradation, such as ester, peptide, anhydride, orthoester, phosphazine and phosphoester bonds. The time required for a polymer to degrade can be tailored by selecting appropriate monomers. Differences in crystallinity also alter degradation rates. For relatively hydrophobic polymers, actual mass loss typically only begins when the oligomeric fragments are small enough to be water soluble. Thus, initial polymer molecular weight influences the apparent degradation rate.

Degradability of the formed polymer depends on the types of acid or acids (or other degradable monomer) used, as well as the types of polyol or polyols used. Common polyols useful in the present invention are aliphatic or substituted aliphatic alcohols containing a minimum of 2 hydroxyl groups per molecular chain. Since a liquid is desired, the polyols are of relatively low molecular weight, for example less than about 20,000 D, more preferably less than about 10,000 D, more preferably less than about 8000 D, and typically in the range of about 1000 to about 5000 D. The preferred polyols contain fewer than 8 hydroxyl groups, and more preferably have an average number of hydroxyls per polyol molecule that is at least slightly greater than 2, to permit crosslinking, but is preferably less than about 4 and more preferably is less than about 3. Alternatively, polyester and polyether polyols or mixtures of these are useful. Generally, hydrophilic polyols or polyol components will accelerate biodegradation by swelling the formed polymer whereas hydrophobic polyols tend to strengthen the formed polymer and delay polymer loss.

Suitable alcohols includes adonitol, arabitol, butanediol, 1,2,3-butanetriol, dipentaerythritol, dulcitol, erythritol, glycerol, hexanediol, iditol, mannitol, pentaerythritol, sorbitol, sucrose, triethanolamine, trimethylolethane, trimethylolpropane and combinations of ethylene and propylene oxides with various amines.

Polyether polyols suitable in the present invention are readily available and include polyoxyethylene homopolymers, and random and block copolymers of oxyethylene (ethylene oxide) with propylene oxide, butylene oxide, trimethylene oxide, and other compounds forming polyether polyols. The percentage of ethylene oxide will generally be in the range of about 70% or more, on a molar (number) basis; e.g., a preferred polyether polyol has 75% of the subunits derived from ethylene oxide, and 25% from propylene oxide. The polyether polyols used in the invention are often diols, but a certain proportion of triols will typically be used to provide crosslinking.

A preferred degradable polyol composition includes a trifunctional hydroxy acid ester and linear polyoxyethylene glycol system. In the prepolymer, the trifunctional hydroxyester acts as the crosslinking agent linking together the polyoxyethylene glycol chains. In the body, chemical action degrades the ester leaving essentially linear chains of polyether polyol that are free to dissolve or degrade. In this system, increasing the percentage of degradable crosslinker increases rigidity, and resistance to swelling and solvation in the crosslinked polymer.

Other polyol systems include hydroxy acid esterified linear polyether and polyester polyols optionally blended with a low molecular weight alcohol. Similarly, polyester and polyether triols esterified with hydroxy acid are useful.

The prepolymer of the adhesive of the invention is formed by capping the polyols with polyisocyanate, preferably a diisocyanate. Preferable isocyanates have the form R(NCO)x, where x is 2 to about 4, and R is an organic group. Suitable isocyanates include: 9,10-anthracene diisocyanate, 1,4-anthracenediisocyanate, benzidine diisocyanate,4,4′-biphenylene diisocyanate, 4-bromo-1,3-phenylene diisocyanate, 4-chloro-1,3-phenylene diisocyanate, cumene-2,4-diisocyanate, cyclohexylene-1,2-diisocyanate, cyclohexylene-1,4-diisocyanate, 1,4-cyclohexylene diisocyanate, 1,10-decamethylene diisocyanate, 3,3′dichloro-4,4′-biphenylene diisocyanate, 4,4′diisocyanatodibenzyl, 2,4-diisocyanatostilbene, 2,6-diisocyanatobenzfuran, 2,4-dimethyl-1,3-phenylene diisocyanate, 5,6-dimethyl-1,3-phenylene diisocyanate, 4,6-dimethyl-1,3-phenylene diisocyanate, 3,3′-dimethyl 4,4′diisocyanatodiphenylmethane, 2,6-dimethyl-4,4′-diisocyanatodiphenyl, 3,3′-dimethoxy-4,4′-diisocyanatodiphenyl, 2,4-diisocyantodiphenylether, 4,4′-diisocyantodiphenylether, 3,3′-diphenyl-4,4′-biphenylene diisocyanate, 4,4′-diphenylmethane diisocyanate, 4-ethoxy-1,3-phenylene diisocyanate, Ethylene diisocyanate, Ethylidene diisocyanate, 2,5-fluorenediisocyanate, 1,6-hexamethylene diisocyanate, isophorone diisocyanate, lysine diisocyanate, 4-methoxy-1,3-phenylene diisocyanate, methylene dicyclohexyl diisocyanate, m-phenylene diisocyanate, 1,5-naphthalene diisocyanate, 1,8-naphthalene diisocyanate, polymeric 4,4′-diphenylmethane diisocyanate, p-phenylene diisocyanate, 4,4′,4″-triphenylmethane triisocyanate, Propylene-1,2-diisocyanate, p-tetramethyl xylene diisocyanate, 1,4-tetramethylene diisocyanate, toluene diisocyanate, 2,4,6-toluene triisocyanate, trifunctional trimer (isocyanurate) of isophorone diisocyanate, trifunctional biuret of hexamethylene diisocyanate, and trifunctional trimer (isocyanurate) of hexamethylene diisocyanate

Another approach to creating an in situ polymerizing liquid that biodegrades in the body is to graft the polyol onto a biodegradable center. Suitable polymers for inclusion as center molecules are described in U.S. Pat. No. 4,838,267 to Jamiolowski et al. . They include alkylene oxalates, dioxepanone, epsilon-caprolactone, glycolide, glycolic acid, lactide, lactic acid, p-dioxanone, trimethylene carbonate, trimethylene dimethylene carbonate and combinations of these.

The center molecule may be a chain, a branched structure, or a star structure. Suitable star structures are described in U.S. Pat. No. 5,578,662 to Bennett et al. Isocyanate capped alkylene oxide can be reacted with these molecules to form one or more extended chains. The ends of these chains can therefore participate in crosslinking with other centers or bond to tissue.

Relatively hydrophobic center molecules such as those listed in U.S. Pat. No. 4,838,267 will tend to form rigid solids upon polymerization. Therefore, it is generally preferable to use a relatively hydrophilic polyol, such as one containing a polyalkylene oxide, for example with at least about 80% alkylene oxide, in the final polymerized structure. Furthermore, the alkylene oxide is preferably comprised of at least about 70% ethylene oxide.

These criteria ensure that the polymerized product will flexible enough to prevent stress localization and associated tissue bond failure. Furthermore, star molecules in general are not preferred as major components in the composition because they contain numerous branches. More numerous branching of the center molecule is associated with higher liquid viscosity. Furthermore, highly branched prepolymers will form polymerized products more slowly and with higher modulus. For example, U.S. Pat. No. 5,578,662 quotes a cross-linking reaction time of 5 minutes to 72 hours. Both of these characteristics are undesirable when the prepolymer is intended as a surgical adhesive or sealant.

### Absorbable Compositions

Absorbable prepolymer systems can be composed of discontinuous (solid) and continuous (liquid) parts. The solid part may be absorbable or may not be absorbable. One of the simplest forms of an absorbable implant is one that mechanically breaks into small pieces without appreciable chemical modification. Fracture of an implant can be seeded or propagated by the placement of hard centers in the polymer during formation.

A mixture of the liquid polymer of the present invention with calcium triphosphate particles will, after exposure to fluids or tissue, polymerize into an elastic solid containing inelastic particulate. Movement of the surrounding tissue will deform the elastic implant. Since the particulate cannot deform, stress will localize around these centers and cracks will begin to propagate from these centers. In this way, the rate of disintegration and size of the disintegrated parts can be controlled by varying the particulate size, the modulus of the formed continuous polymer, and the density distribution of the particulate.

Suitable non-absorbable solids are well known and include, among others, calcium triphosphate, calcium hydroxylapatite, carbon, silicone, Teflon, polyurethane, acrylic and mixture of these. Absorbable solids are well known and include glycolic acid, glycolide, lactic acid, lactide, dioxanone, epsilon-caprolactone, trimethylene carbonate, hydroxybutyrate, hydroxyvalerate, polyanhydrides, and mixtures of these. These two-phase systems are not excluded in the present invention, but are also not preferred since the fractured implant particles may not themselves decompose or dissolve.

Another type of absorbable prepolymer liquid can be composed of two mechanically mixed continuous parts. For example, one part may be absorbable and the other not. Consequently, the absorption of one part results in the mechanical disintegration of the implant. Absorbable components may include liquid forms of cellulose ether, collagen, hyaluronic acid, polyglycolic acid, glycolide and others well known in the art. These systems are not excluded in the present invention, but are also not preferred for the reasons stated above.

Absorbable prepolymer systems can be constructed from hydrophilic polyols, such as polyethylene glycol. Polymerized isocyanate capped polyethylene glycol can either swell and mechanically disperse or dissolve, depending on the density of cross links between polyethylene glycol chains.

The polyol need not be entirely absorbable. For example, polymerized isocyanate capped random coblock polymers of polyethylene oxide and polypropylene oxides can be made absorbable by reducing the trifunctionality of the polyol. As crosslinking declines with decreased trifunctionality, the crosslinked implant gradually becomes friable.

### Wound Healing Compositions

The liquids described in this invention can be used to treat wounds. For example their adhesive quality can bring surfaces together and hold them together to promote healing. Also, the material can be coated over a damaged surface to prevent fluid leakage and to promote healing. Also the liquid can be functionalized to promote healing, either by providing a pharmaceutical additive or by adding charge to the polymer. The placement of charge on a polymer in contact with tissue can promote wound healing.

These curative charges can be induced on the capped end of the polymer. For example, addition of diethylethanolamine results in formation of positively charged diethylaminoethyl groups on the polymer. Conversely, a negative charge may be induced by reacting the end capped polymer with carboxymethanol which forms carboxymethyl groups on the polymer.

### Anti-Adhesion Compositions

Edlich et al in the Journal of Surgical Research, v. 14, n. 4, April 1973, pp 277-284 describes the results of applying a topical solution of 10% ethylene/propylene oxide copolymer to wounds. Reduced inflammatory response at the wound was found for copolymer solutions containing ethylene oxide:propylene oxide in the ratio of 4:1. Inflammation is known to be associated with adhesion formation around surgical sites.

One of the applications of the present invention is surgical repair of tissue. The polymer of the present invention is preferably comprised of an isocyanate-capped and subsequently crosslinked structure with a polyethylene/polypropylene oxide (PEPO) backbone. Under biodegradation or absorption of the in situ formed polymer, essentially whole chains of PEPO are released into the body. The decomposition of the implant provides for a continuous supply of PEPO which can serve as an anti-adhesion agent during wound healing. The released polyoxyalkylene block copolymers are eventually excreted in a non-metabolized form.

Further increases in the rate of release of PEPO can be made by adding PEPO directly to the prepolymer of this invention, at the time of application of the prepolymer to the tissue site. The result is a prepolymer which will spatially trap PEPO as a hydrogel, such that the action of water in the body is both to initiate crosslink formation between the isocyanate capped polyols and tissue as well as form a hydrogel with the un-capped PEPO.

The three dimensional structure of the resulting crosslinked implant holds the PEPO hydrogel by physical crosslinks, such as hydrogen bonds, rather than covalently. Since these bonds are reversible, thermodynamic considerations will drive the PEPO to slowly elute from the implant. This action will decrease the volume of the implant, without breaking the bonds of the crosslinked structures. Thus, an absorbable implant is formed having potentially both absorption and decomposition pathways to volume loss.

There are three basic approaches to preventing post-surgical adhesions. The first involves the use of a lubricious liquid placed around the surgical site to create a situation termed in the prior art as “hydroflotation”. Hydroflotation prevents tissue surfaces from coming into contact and forming adhesions. The second involves the placement of a solid layer between tissues surfaces to separate them. The third involves the adherence of a separating layer to tissue to both prevent contact between tissue layers and to seal damaged tissue sites. The release of biologically active fluids from wounded tissue is known to promote adhesion formation.

It is clear from the above description of the PEPO supplemented prepolymer that all three of these anti-adhesion mechanisms are uniquely provided in this embodiment of the present invention.

The PEPO-supplemented adhesive has an additional property which can be very beneficial. The adhesive crosslinks in the presence of small amounts of water, and can readily crosslink faster than the PEPO or other polyalkylene oxide polymers can disperse. As a result, the entry of water into the crosslinked material is inhibited, and the material does not swell, or swells slowly, in biological fluids. This is advantageous for coated devices, for example coated meshes, both for immediate ease of handling during application, and for prevention of stress on hollow structures as the gel swells.

### Adhesive Synthesis Examples

All reagents were obtained from commercial catalogs, for example Aldrich Chemical.

### Example 1

### Biodegradable In-Situ Polymerizing Implant (Lactated Trimethylolpropane)

A commercial polyether polyol, UCON 75-H450, obtained from Dow Chemical Co., and described as having a molecular weight of about 978 D, and a composition of about 25% propylene oxide monomers and 75% ethylene oxide monomers, and as being a diol, was dried by heating at 82 deg. C. for 6 hours at 2 Torr of pure nitrogen flowing at 1 cubic foot per hour. Trimethylolpropane (TMP) was lactated by mixing 269 g of TMP with 1486 g of 85% lactic acid and heating at 2 Torr of pure nitrogen flowing at 1 cubic foot per hour for 2 hours at 110 deg. C. and subsequently for 24 hours at 125 deg. C. 1244 g of dried UCON 75-H-450 was mixed with 133 g of lactated TMP and heated at 82 deg. C. under nitrogen flow of 1 cubic foot per hour for 8 hours. Toluene diisocyanate (TDI) was subsequently added to obtain a theoretical NCO content of approximately 3.0 (i.e., enough to cap all of the free hydroxyl ends of the polyether polyol after it was bonded to the TMP or lactide by dehydration; typically, a few percent of the isocyanate is left over, c.f. US publication 2003-0135238), and heated at 82 deg. C. under nitrogen flow of 1 cubic foot per hour for 24 hours. The resulting liquid tissue adhesive was stored in a vessel or ampoule sealed to prevent uptake of moisture, and stored at room temperature in the dark or under artificial light.

### Example 2

### In Situ Polymerizing Implant (Pure Polyethylene Glycol)

Certain polyols are highly hydrophilic, such a polyethylene glycol (PEG), and will swell and subsequently dissolve in the body. Carbowax 1000, a 1000 MW PEG, was dried according to the procedure of Example 1. 1269 g of dried Carbowax 1000 was mixed with 53.9 g of TMP and heated at 82 deg. C. for 8 hours under nitrogen flow of 1 cubic foot per hour. Subsequently, TDI was added to obtain a theoretical NCO content of 2.8, and the mixture was heated at 82 deg. C. for 24 hours under a nitrogen flow of 1 cubic foot per hour to obtain a liquid tissue adhesive. Storage was as in Example 1.

### Example 3

### In Situ Polymerizing Implant (Reduced Trifunctionality)

The polyol was trifunctionalized to a reduced extent by reducing the ratio of TMP used to the number of polyols on the polymers. For example, 1902 g of UCON 75-H-1400, a 25:75 copolymer of EtO and PrO, molecular weight 2500 D (Dow), was dried and mixed with 34 g TMP and heated under a 2 Torr nitrogen flow of 1 cubic foot per hour for 8 hours at 82 deg. C. After the TMP is consumed, TDI is added in sufficient quantity to obtain a theoretical NCO content of 2.5 and heated at 82 deg. C. under a nitrogen flow of 1 cubic foot per hour for 24 hours, and stored as described in Example 1.

### Example 4

### Biodegradable In-Situ Polymerizing Implant (Lactated Diol)

2450 g of dried UCON 75-H-450 was mixed with 900 g of 85% lactic acid and heated at 85° C. under a nitrogen flow of 1 cubic foot per hour for 8 hours. 1274 g of lactated UCON 75-H-450 was mixed with 24 g of TMP and 490 g of UCON 75-H-450 and dried. After dry, the mixture was reacted for a further 4 hours at 85° C.

### Example 5

### Biodegradable In-Situ Polymerizing Implant (Lactated Triol)

UCON LB-65 (Dow; MW XXXX D; EtO:PrO=XXXX) was dried and reacted with TMP to form a triol of mean molecular weight 1300. 390 g of dried triol was added to 190 g of 85% lactic acid and heated to 120 deg. C. under a 2 Torr nitrogen flow of 1 cubic foot per hour for 16 hours. 150 g of dried lactated triol was reacted with 109 g of TDI at 82 deg. C. for 8 hours under a nitrogen flow of 1 cubic foot per hour, and stored as described above.

### Example 6

### Absorbable in Situ Polymerizing Implant (Hypothetical)

An absorbable implant could be made from any of the adhesive polymers of Examples 1-5, if a solid particulate is added to the composition, for example, a solid particulate composed of calcium carbonate. The particulate will preferably comprise approximately 25% by volume of the total composition volume. Upon implantation, typically after dilution (at the time of implantation) with about 1 vol. of sterile saline or other sterile biocompatible fluid, the mixture is expected to cure into an adherent implant, which will gradually disintegrate as the calcium carbonate absorbs water.

### Example 7

### Biodegradable in Situ Polymerizing Pharmaceutical Reservoir (Hypothetical)

A degradable implant could be made from any of the adhesive polymers of Examples 1-6 that further comprises physiologically or pharmaceutically active ingredients (drugs, chemicals and the like) added to the composition. For example, an aqueous antibacterial is added to the prepolymer, and can be released into the body by diffusion from the implant, or as the polymerized implant disperses. If the active ingredient will react with an isocyanate, it can be pre-encapsulated in an excipient polymer that is not significantly reactive with isocyanate groups, such as polyethylene oxide, polyvinyl pyrrolidone, and other approved excipients not containing hydroxyl or amine groups except at the ends of polymers.

### Example 8

### Fast-Curing Tissue Adhesive

In this example, an isocyanate-terminated diol was trifunctionalized to yield a fast curing tissue adhesive. Fast curing adhesives cure within 5 minutes when used neat and applied to tissue. Slow adhesives cure after this time, generally 5 to 10 times longer. The type and amount of isocyanate used was 270.26 g of toluene diisocyanate (TDI). A suitable TDI for a fast cure is Rubinate, a mixture of 80% 2-4 and 20% 2-6 isomers (Huntsman Chemical); other brands of TDI are likely to prove suitable. The type and amount of diol used was 870.53 g of Ucon 75-H-450. The type and amount of triol used was 9.21 g of trimethylol propane (TMP). The theoretical target for completion of the diol termination steps is % NCO=4.55%. The theoretical target for completion of the trifunctionalization step is % NCO=3.76%. Final temperature before TMP addition was 50 deg. C. The NCO levels at 25 hrs 4.78% and at 75 hrs 4.55%. Then the TMP was added at hour 76. The final NCO of % NCO=3.67% was reach at hour 100. The viscosity at 31 deg. C. was 24,500 centipoise.

The above tissue adhesive forms a tissue bond of strength 4 lb/in2 in tension and about 25 lbs/in2 in shear, measured by adhering a strong cloth to a tissue surface (beef chuck roast) and tearing the cloth from the roast in an Instron tester.

Details of the preparation of this material, which is presently preferred for many applications, and of other presently preferred materials, are found in co-pending priority document U.S. 60/557,314, “Surgical Adhesive Formulations and Methods of Preparation”, by Michael T. Milbocker, which is hereby incorporated by reference to the extent permitted.

### Example 9

### Absorbable Adhesive Without Chemical Decomposition

React 824.93 g of UCON 75-H-1400 with 171.29 g IPDI at 60 deg. C. until isocyanate level reaches 3.1% NCO. Add 12.49 g TMP and react at 60 deg. C. until isocyanate level reaches 2.0% NCO. The resulting adhesive has a viscosity of 29 Kcps (kilocentipoise) at 31.8 deg. C. When reacted with water vapor, the prepolymer forms a hydrogel. When placed in several volumes of water, the hydrogel disperses in about 7 days. Longer dispersal times can be achieved by increasing the TMP amount, achieving dispersal times exceeding 2 years.

### Example 10

### Low Viscosity Fast Cure Adhesive

React 700.00 g of UCON 75-H-1400 with 113.38 g TDI at 60 deg. C. until isocyanate level reaches 2.95% NCO. Add 6.26 g TMP and react at 60° C. until isocyanate level reaches 2.21% NCO. The resulting adhesive has a viscosity of 26 Kcps at 31.8 deg. C.

### Example 11

### Mesh Coating Composition and Coated Mesh

To prevent differential expansion of a mesh substrate and a hydrogel coating, a polyether diol was added to the adhesive prepolymer at the same time that water is added to initiate polymerization. For example, when ready to coat a mesh, for example when coating polypropylene mesh (Surgipro, USS), equal parts of the adhesive of Example 10 and of dried UCON 75-H-450 polyether polyol were mixed together. Then that mixture was mixed with water or saline at a 2:1 ratio. The mesh was laid on a flat, non-reactive (metal) surface and the water-diluted adhesive: polyol mixture was applied and allowed to cure. The composition had a cure time of about 2 minutes, and could be worked into the pores of the mesh with a straightedge, and the surface leveled with a straightedge or blade, before the composition sets. Alternatively the composition can be sprayed onto the mesh, or applied between two opposing rollers. The resulting coated mesh remained flat when placed in water, in contrast to a similar coated mesh without the added polyol.

### Applications of the Adhesive to Treat Various Conditions

### 1. Urinary Stress Incontinence

Stress incontinence is the involuntary loss of urine due to a sudden rise in intra-abdominal pressure. In women, the urethral continence mechanism is governed by four factors: 1) urethral closing pressure, 2) urethral length, 3) urethrotrigonal anatomy, and 4) the efficiency with which intra-abdominal pressure is transmitted to the urethra

The urethral closing pressure is largely regulated by smooth and striate muscles. There is a lesser contribution from nonmuscular factors such as the health of the submucosal vascular plexus, the elastin and collagen content of the urethral tissue, and sphincter effects of the mucosa. It has been suggested that the orientation of the urethra with respect to the bladder can significantly impact the sphincter action of the mucosa. While it has been observed that a short urethra can be correlated with reduced continence in females, a short urethra alone is not predictive of incontinence. A short urethra in combination with other factors such as mobility of the bladder and urethra can produce incontinence.

In the hypermobile case, a loss of normal urethrotrigonal anatomy may occur. For normal anatomy, the bladder base should lie above the level of the inferior ramus of the symphysis, and straining should not result in a descent of more than 1.5 cm. The urethrotrigonal alignment should form an angle less than 100 degrees, and the urethral axis should be approximately 35 degrees from vertical. When this normal anatomical configuration is maintained, then transmission of intra-abdominal pressure to the intra-abdominal portion of the proximal urethra achieves continence. When the urethral axis is altered the rotational descent will drop the proximal urethra and bladder base and the transmission of intra-abdominal pressure will serve to open the proximal urethra. Vesicourethropexy procedures aim to correct the anatomical alignment by anteriorly elevating the bladder neck and also by elongating and narrowing the proximal urethra. These are usually done by suspending the urethra, for example with tapes or slings, to minimize its mobility. While these approaches are effective, they are also invasive. A minimally invasive procedure is needed which corrects anatomical misalignment. Moreover, procedures that do not subject the urethra to erosion would be preferable to some present procedures.

Several approaches are envisaged to use the adhesives and methods of the invention to augment or support the urethra and/or bladder to treat incontinence. In the present invention, liquid adhesive compositions capable of bonding tissue while forming a solid in situ are used to augment or support the urethra and/or bladder to treat incontinence. One approach is to provide a mechanism for fixing the urethra to transvaginal tapes by incorporating the adhesive in the mesh structure of the tape. A second approach is to use the injectable polymer alone to augment and fix a portion of the urethra in much the same way mid-urethra suspension or slings are used.

In particular, in one approach the present invention comprises an in situ polymerizing solution, a suspension means, and optionally a protective sheath. Devices useful in conjunction with a bonding liquid polymer are porous implantable tapes such as the SPARC™ Female Sling System (American Medical Systems), and similar devices and kits known in the art. Accordingly, the present invention may, as described in U.S. Pat. No. 6,334,446, employ thin, curved stainless steel needles advanced through two tiny incisions above the pubic bone to a vaginal incision below the urethra. A porous polypropylene sling, contained in a plastic passing sheath, is then attached to the needles. In the method of the invention, liquid polymer is injected into the space enclosed within the plastic sheathing. The assembly is then passed through the needle tunnel. Following correct sling placement the sheath is removed and the polymer is exposed to tissue. The sling tension is adjusted to provide support for the urethra and held in place until the polymer cures.

Alternatively, in the method of the invention the sheath can be eliminated and the adhesive placed directly on the sling material, before or preferably after the sling is positioned under the urethra. In another aspect of the invention, the adhesive polymer can be pre-applied to a sling, and optionally foamed while curing, to provide a resilient pad section on the sling for support of the urethra. In another aspect, the sling itself can be made of the polymerized adhesive of the invention.

Bladder slings can likewise be padded with cured adhesive, and/or can be made of degradable materials if required. Further, any sling or similar device can optionally be adhered to surrounding tissue with polymerized adhesive.

While the above procedure corrects for urethral hypermobility, in some instances a sling is not required. In fact, in some patients urethral mobility can aid in achieving continence, provided that the urethra is immobilized at a point. For example, it has been observed that mobility of the proximal urethra can predict the objective outcome of a tensionless suburethral tape procedure. The more the proximal part of the urethra moves under stress, the better the continence achieved by placing the sling under the mid urethra.

In another aspect of the present invention, the adhesive of the invention is used to immobilize the mid-urethra to take advantage of urethral mobility to avoid leakage. The mid-urethra is immobilized by injecting 1-3 cc of liquid polymer outside the urethra, in a manner such that a bond is formed between the urethra and surrounding tissue, thus reducing the mobility of the urethra at the injection point. This procedure mimics the tension-free tape procedure since it does not support or lift the urethra Where applicable, it is a simple procedure to execute, and the absence of a “hard” material such as a sling in contact with the urethra will tend to prevent erosion of the urethra. If reinforcement of the bond between the urethra and the adjacent tissue is required, a tape or sling may be glued to the outside of the cured adhesive surrounding the urethra.

### 2. Herniation

There are a variety of injuries or defects in which a first tissue protrudes through a defect into a location which is normally closed to the first tissue and/or occupied by a second tissue. A defect in the pelvic floor may be created during childbirth, for example a vesicovaginal fascia, or by injury to the pelvic floor, A defect that results in herniation of the bladder is called a cystocele. Herniation of tissue is similar in rectoceles, enteroceles and enterocystoceles. A rectocele is herniation of the rectum. An enterocele is herniation of the intestine through the rectovaginal or vesicovaginal fascia An enterocystocele is herniation of both the bladder and intestine. An inguinal hernia is a penetration of another organ, typically the intestines, into the inguinal canal. An indirect inguinal hernia is a condition, typically resulting from failure of the inguinal canal to close, in which the peritoneal lining extends into the inguinal canal, forming a sac in which intestinal tissue becomes trapped.

These conditions are treated by repositioning the protruding tissue to its normal physiological position. Typically, repositioning is followed by closure of a defect and/or fixation of tissue to prevent recurrence. The compromised tissue layer that resulted in the aneurized condition may require reinforcement. This is sometimes accomplished with a synthetic mesh. Localization of the mesh requires sutures, which may result in damage to adjacent tissue. Placement of the mesh may require large incisions in the vagina, or elsewhere, which may later cause discomfort. Thus, there is a need for a device and method that reduces the number of sutures or staples needed to repair a herniation, or eliminates them. It is further desirable to eliminate the use of a mesh, or to minimize such use, and to reduce the number or size of the incisions required to place the mesh.

2A. Reduction of an Inguinal Hernia with tissue Adhesive.

There are two principal types of inguinal hernia An indirect inguinal hernia is a condition in which an extension of the peritoneal lining (processus vaginalis) extents into the internal inguinal ring and into the inguinal canal forming a sac in continuity with the peritoneal cavity. This can result in a hernia when part of the intestine falls into the sac. As a result, the intestine can become trapped, forming an incarcerated hernia requiring emergency surgery. This type of inguinal hernia occurs most often in infants and children, and less commonly in adults. The indirect inguinal hernia differs from the common adult direct inguinal hernia that results from a weak spot in the floor of the inguinal canal that allows the peritoneum and intestine to directly enter the canal. A direct hernia, also simply called a hernia, occurs when abdominal tissues push through the inguinal canal, which is normally closed after birth. This is in effect a rupture of a previous closure. An indirect hernia occurs when abdominal tissues protrude through the inguinal canal, when it has not yet closed, or has not closed properly.

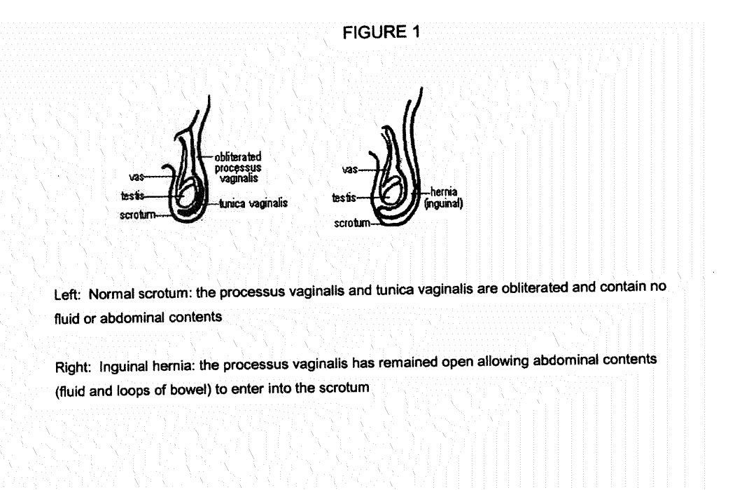

FIG. 1 is a schematic illustration of an indirect or direct inguinal hernia when positioned in the scrotum. The left diagram shows the normal situation. The right diagram illustrates an open inguinal passage containing a sac of peritoneum. Repair of a hernia may consist of removing any tissue found in the sac, and then restoring closure or converting the sac to be closed, as in the left diagram.

### Surgical Approaches

There are usually no symptoms that a child has an inguinal hernia until abdominal organs are forced into the sac. Swelling can sometimes be seen in the groin area or scrotal sac when a baby is crying or straining or when an older child coughs, strains or stands for a long time. If the bulging can be gently pressed back into the abdomen, the hernia is known as reducible. If a loop of the intestine is forced into the sac, the hernia is then known as incarcerated (irreducible). An infant or a child will show signs of irritability, loss of appetite, tenderness and swelling of the abdomen or have trouble having a bowel movement. With incarceration, the intestines have entered the sac and are being strangled. This portion of the intestines could die. The main treatment for inguinal hernia is surgery to remove the hernia sac and tie off the communication at the level of the internal inguinal ring. This surgery is called an indirect inguinal herniorraphy.

The present invention allows improved, simplified and/or less invasive procedures to be used repair of hernias. In one aspect, the invention allows for the repair of a hernia using an adhesive of the invention to seal incisions and to hold repair meshes in position without requiring stitches. In another aspect, a non-incarcerated indirect inguinal hernia can be closed and sealed using the adhesive of the invention. In the case where the hernia is reducible, the herniated tissue is repositioned to a normal position with hand pressure and the region between the intestine and peritoneum is reinforced with adhesive and the opening in the inguinal canal is sealed with adhesive. In the case of incarcerated hernia, the intestine is repositioned and the opening in the inguinal canal is paved with adhesive reinforced with mesh or flock.

In one embodiment of the present invention, a laparoscope is introduced through the belly button of a patient diagnosed with an inguinal hernia, after reduction to remove tissue in the inguinal canal, if needed. A catheter is placed in the working channel of the scope, which is capable of delivering a liquid prepolymer. The liquid prepolymer, when in contact with tissue, absorbs fluids and polymerizes with the proteins in the tissue to reduce fluid accumulation and to form a solid elastomeric plug bonded to the surrounding tissue. The pouch formed by the peritoneum intruding into the inguinal canal provides a closed space into which the adhesive can be delivered and localized. Delivery of the adhesive into the pouch reduces the volume of the pouch by pulling it together and sealing the pouch, thereby preventing future fluid communication (hydrocele) and recurrence of hernia.

### Example 12

### Hernia Repair in a Model System

The efficacy of a hydrophilic isocyanate terminated prepolymer as a tissue approximating fluid was tested using a 3-branch polyol terminated with TDI, prepared as described in Example 8. An experiment was conducted to demonstrate that a tissue adhesive of the present invention is capable of removing fluid present in a living tissue cavity, and thus capable of bringing into contact tissue layers that are pathologically separated by a fluid accumulation. The invention accomplishes this goal by incorporating fluid, principally water, into the formed in situ polymerized mass during polymerization. This prevents the re-intrusion of fluid or of exogenous tissue into the cavity, thereby repairing it in a manner not requiring extensive surgery.

Young Yorkshire pigs possess an anatomical structure similar to an indirect inguinal hernia occurring in humans. The condition is characterized by an extension of the peritoneal lining into the internal inguinal ring and into the inguinal canal forming a sac in continuity with the peritoneal cavity. In humans, this can result in a hernia when part of the intestine falls into the sac. As a result, the intestine can become trapped forming an incarcerated hernia requiring emergency surgery.

A 20 kg Yorkshire Pig was anesthetized and placed in the supine position. An incision was made just above the inguinal canal and the sperm cords exposed. The full length of the inguinal canal was exposed and a fluid accumulation was observed causing this space to bulge noticeably. Prepolymer of the present invention (example 8) was injected into the space and massaged to incorporate prepolymer and fluid. The volume in the inguinal canal was seen to decrease over a period of 5 minutes, whereby the inguinal canal was inseparably closed and the layers of tissue forming the canal wall brought into intimate contact, such that the appearance of the result was that the inguinal canal was actively evacuated.

The advantage of this approach over direct fluid evacuation is that when one attempts to evacuate at the level of the inguinal ring, valving occurs due to the tortuosity of the canal geometry, and thus fluid at distal points is not fully evacuated. Moreover, the procedure of the invention permanently closes the whole length of the canal. This cannot in general be achieved with sutures, since fluid readily passes beyond any suture line meant to maintain the inguinal canal in an evacuated state. Additionally, the hydrophilic nature of the prepolymer is such that it both actively pulls free water into it and is pulled into tissue containing water, thus effectively incorporating water and polymer in the tissue and eliminating the fluid volume responsible for maintaining the cavity.

A non-laparoscopic approach was taken in this experiment, for convenience and because this was the first test of the procedure. Clearly, a similar procedure in humans could be done laparoscopically, using standard techniques.

### Example 13

### Implantation of Surgical Mesh with Adhesives of the Invention

The following experiment demonstrates the efficacy of the adhesive in the immobilization of implanted meshes in a model system. Twelve Wilshire pigs were implanted with three types of mesh. The first type was a SurgiPro (Chicago, Ill., USA) plug and patch mesh set consisting of a plug formed from mesh to be inserted into the herniation and an overlaying mesh sheet (4×10 cm). The second type was a polypropylene mesh (SurgiPro) measuring 10×10 cm. The third type was a polyester mesh measuring 10×10 cm. The glue used was the material of Example 8.

The Plug and Patch was implanted by filling a surgically formed abdominal defect with the plug and gluing the patch over the filled defect. A 0.1 cc volume of surgical adhesive was place as a dot midway along each edge of the patch. Four 0.1 cc applications were applied in total per patch. The polypropylene and polyester meshes were implanted similarly, with 20 applications of 0.1 cc of glue uniformly distributed on the perimeter of each mesh.

Controls consisted of side-by-side mesh implantations using suture. Each glue application in the glue fixed meshes was replaced by a suture placement in the control meshes. Two animals received 3 cc of glue by itself, applied in the groin region. All mesh positions were identified by two orthogonally placed sutures 1 cm distant from the mesh.

The animals were survived 90 days. At necropsy the meshes were exposed and their dimensions, position with respect to the suture marker, and mesh adherence to tissue were measured. Histology was taken of the liver, kidney, and adjacent lymph nodes, as well as tissue at the interface of the mesh with tissue.

Marked inflammation was identified for polypropylene mesh, as is known. Moderate inflammation was identified for polyester mesh. Minimal to no inflammation was found in the region where surgical adhesive was placed alone, without mesh. Quantitative results included:

### Migration of Implant (in cm)

| Plug and Patch | Glue | 1.5 +/− 1.3 cm |
|---|---|---|
| | Suture | 2.0 +/− 1.2 |
| Polypropylene | Glue | 2.3 +/− 0.8 |
| | Suture | 2.8 +/− 2.8 |
| Polyester | Glue | 2.3 +/− 0.3 |
| | Suture | 2.0 +/− 1.6 |

### Pull Force to Remove Mesh (Newtons)

| | | |
|---|---|---|
| Plug and Patch | Glue | 64 N |
| | Suture | 90 N |
| Polypropylene | Glue | 134 N |
| | Suture | 166 +/− 39 N |
| Polyester | Glue | 144 +/− 40 N |
| | Suture | 195 +/− 35 N |

### Shrinkage of Mesh (Area)—Final Area as Percent of Original

| | | |
|---|---|---|
| Plug and Patch | Glue | 92% +/− 9 |
| | Suture | 88% +/− 13 |
| Polypropylene | Glue | 78% +/− 6 |
| | Suture | 84% +/− 15 |
| Polyester | Glue | 86% +/− 9 |
| | Suture | 92% +/− 7 |

This data indicates that the adhesive-affixed meshes and patches shrank and “migrated” (due to shrinking) about the same as the suture-affixed meshes, and required somewhat less tensile force to be detached from the tissue. Application of the adhesive will be significantly simpler than application of sutures in laparoscopic surgery. In addition, there may eventually prove to be less pain associated with the contraction of the meshes in the adhesive-affixed meshes.

### Example 14

### Using Flock in Adhesive in Place of a Mesh

Adhesive of the invention (from Example 8 above) was used to reinforce tissue in two different ways. In one mode, the adhesive was used to prepare a flock/adhesive composite material which could be glued or stapled to tissue to reinforce it. In another mode, the use of adhesive to reinforce a mesh was examined.

The flock used was 0.30-2.5 DPF natural polyester obtained from Cellusuede Products, Rockford Ill. (USA). Flock was mixed with a toluene diisocyanate based adhesive of, Example H, at ratios of 1:3 (25% flock) by volume, 1:1 (50%) and 3:1 (75%). The mixture was diluted with a volume of acetone (or, in other experiments, toluene) equal to the volume of adhesive prepolymer. This mixture was poured into dishes to a fixed depth and allowed to cure by absorption of atmospheric moisture while solvent evaporated, for about a week. The final product was a translucent circular patch material. The volume of flock was determined by packing it into a syringe and compressing it by hand, and should be considered a qualitative measurement, useful for approximating the effects of flock on polymerized adhesive properties. (The actual weight concentration of the flock in the adhesive/solvent mixture was estimated to be in the range of a few percent or less.)

To determine suture-holding force, a suture (type 2-0 or 5-0) was passed once through the reinforced adhesive, and the force required to pull the suture out (sideways, tearing through the reinforced adhesive patch) was measured in an Instron (brand) mechanical property tester. Once the tear was started, the force was relatively constant. In addition, a strip 1 cm wide was cut from the composite, and the force required to rupture it was measured on the Instron instrument. Table 1 shows the results obtained in a “25% flock” composite, while Tabel 2 shows the required forces at constant thickness and variable nominal percentage of flock. Table 3 shows tearing forces in a mesh coated with adhesive. The mesh was an absorbable hemostat made of oxidized regenerated cellulose (“Surgicel” from Ethicon/Johnson & Johnson).

TABLE 1
Forces (lbs) to tear or rupture at 25% v/v flock

| | 2-0 | 5-0 | |
|---|---|---|---|
| Thickness | Suture | Suture | Rupture |
| 0.4 | 0.5 | 0.5 | 2.7 |
| 0.75 | 1.1 | 0.9 | 4.8 |
| 1.5 | 2.7 | 2.3 | 9.8 |

TABLE 2
Forces at 0.75 mm Composite thickness

| | 2-0 | 5-0 | Max. |
|---|---|---|---|
| % Flock | suture | suture | Force |
| 25 | 1.1 | 0.9 | 4.8 |
| 50 | 2.2 | 1.7 | 8 |
| 75 | 2.1 | 1.8 | 9.1 |

TABLE 3
Forces to tear coated mesh (lb force)

| | 2-0 | 5-0 | |
|---|---|---|---|
| Thickness | Suture | Suture | Rupture |
| 0.5 mm | 0.6 | 0.6 | 1.2 |
| 1 mm | 0.6 | 0.6 | 1.6 |

The forces required to pull out a suture, or to rupture the composite, increase significantly with thickness and with increase in flock concentration, as expected of a composite material. In particular, for constant % flock, maximum force increased linearly with sheet thickness. Incremental increases in maximum force were greatest for low flock density, while maximum force was achieved for highest flock density. Clearly, the inclusion of a flock in a cured film of the adhesive of the invention allows control of the tensile properties of the film. The particular method used for prototype production will require adaptation for mass production, where a continuous mixing process placing water or polyol in the adhesive/flock mixture would be preferred.

### Repair of Rectoceles and Similar Types of Herniation

A defect in the pelvic floor may be created during childbirth, for example a vesicovaginal fascia, or by injury to the pelvic floor, A defect that results in herniation of the bladder is a cystocele. Herniation of tissue is similar in rectoceles, enteroceles and enterocystoceles. A rectocele is herniation of the rectum. An enterocele is herniation of the intestine through the rectovaginal or vesicovaginal fascia. An enterocystocele is herniation of both the bladder and intestine.

These conditions are treated by repositioning the protruding tissue to its normal physiological position. The compromised tissue layer that resulted in the aneurized condition may require reinforcement. This is sometimes accomplished with a synthetic mesh. Localization of the mesh requires sutures, which may result in damage to adjacent tissue. Placement of the mesh often requires large incisions in the vagina, or elsewhere, which may later cause discomfort. There is a need for a device and method that reduces the number of sutures needed to repair a herniation. It is further desirable to eliminate the use of a mesh and/or at least one of the incisions required to place the mesh.

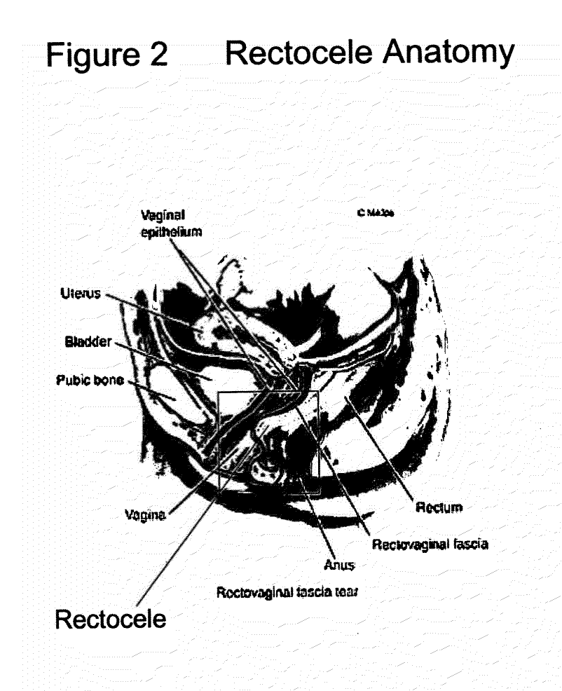

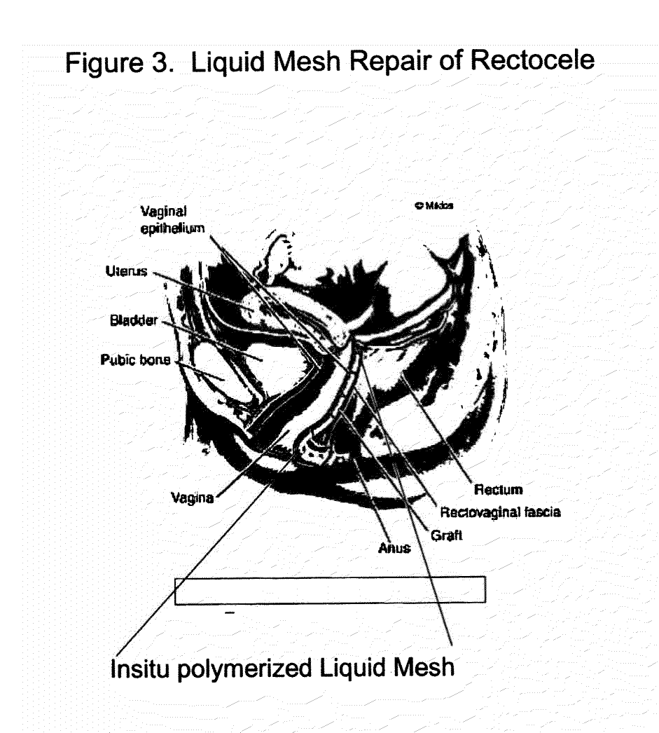

In one embodiment, the method of the invention is treatment for rectocele, and the tissue to be supported is the rectum. The relevant anatomy is illustrated in FIG. 2, and the protrusion of the rectum into the vagina is the rectocele. The lesion as it would appear after being repaired using the adhesive of the invention is shown in FIG. 3.

The standard method for repairing the lesion is, in brief, to make a transverse incision between the anterior rectal wall and posterior vaginal wall. Sharp and blunt dissection is used to separate the posterior wall of the vagina from the rectum. This space is normally dissected to provide a place for a mesh. In the present invention, the liquid adhesive in injected into the floor of this space and the posterior vaginal wall is pulled out and down to reapproximate the space. Polymerization of the adhesive will simultaneously adhere the two planes together, while forming a malleable soft layer that will provide support without sutures or clips.

A more detailed procedure for correcting a rectocele is also described, so that it can easily be understood how an actual procedure using the materials and methods of the invention will be performed. A transverse incision is made in the vaginal epithelium between the anterior rectal wall and posterior vaginal wall. The vaginal epithelium is retracted with Allis clamps. The rectovaginal septum is dissected from the posterior vaginal wall to completely expose the rectovaginal septum defect and protruding anterior rectal wall. In many cases the rectovaginal fascia has completely detached from the iliococcygeal muscles on each side. Using finger pressure the anterior rectal wall is displaced below the rectovaginal fascia to its normal position. Finger pressure is then directed to the left and the left side of the rectovaginal fascia is brought in opposition to the left iliococcygeal muscle. Adhesive of the invention, for instance of Example 8, or containing reinforcing flock as in Example 11, is then placed at the junction to permanently attach the left side of the rectovaginal fascia to the left iliococcygeal muscle. When the adhesive has cured the right side is repaired in similar fashion. Adhesive is then applied over the entire surface of the exposed rectovaginal fascia, taking care to also coat the exposed surfaces of the iliococcygeal muscle laterally and perineal body distally. The vaginal epithelium is then closed over the glued surface and pulled out and down. A pessary or finger pressure may be used to flatten the posterior vaginal wall. The adhesive is allowed to cure. Excess vaginal epithelium is trimmed and the incision is closed.

Having described the procedure for repair of rectocele, it should be obvious that an analogous technique could be used to repair other forms of pelvic floor herniation, including cystoceles, enteroceles and enterocystoceles, as well as direct and indirect inguinal herniations and other types of herniation.

The above repair technique also could be combined with placement of a mesh on the rectovaginal fascia before closing. The closure of particular incisions or areas of rupture may be made entirely by adhesive, or the adhesive may in part be supplemented or replace by sutures, staples, clips, and other mechanical tissue fixation means.

In another aspect of the invention, a method of stabilizing the pelvic floor is described. In this method a continuous layer of adhesive is applied with one part of the layer in contact with the tissue to be reinforced and another part in contact with a supporting structure. Typical supporting structures include fascia, ligament, bone, and muscle. The supporting structure must be located so that when the surgical adhesive cures in the therapeutic position, the forces generated by these supporting structures are sufficient to maintain the therapeutic position of the tissue. In some embodiments the surgical adhesive bonds to the arcus tendinous fascia pelvis. In others, the adhesive bonds to the ileal pectineal muscle group. In still others, the adhesive bonds to the pubococcygeous muscles.

### Other Uses

Example 14 illustrated the use of the adhesive composition to coat a mesh. Because of the nature of the adhesive, no “tie” layer is required. The adhesive could be used to coat other implantable materials as well, using similar procedures. When it is desired to have a non-swelling coating, the use of adhesive compositions containing polyols without isocyanate can provide the required dimensional stability. Because the gradual diffusion of polyether polyol tends to prevent adhesions, and may diminish inflammation and cell ingrowth, it is possible by selective coating to have different regions of an implant adhere to surrounding tissue to different degrees., and/or differing degrees of swelling of the coating. It should be noted that when bonding tissues together, as in the example of rectocele repair in FIG. 3, tissue can grow through a porous mesh and join with tissue on the other side, thereby embedding the mesh and creating a strong bond in the region of repair. If the mesh is made of or coated with the adhesive, the openings should be large enough, or punched to be large enough, to permit this tissue bonding—for example, at least about 0.1 millimeter, and preferably larger.

The invention has been described using examples and anticipated procedures to enable the skilled person to understand the invention. The invention is not limited to the particular examples used to illustrate it, but is limited only by the claims.

1. A method for fixation of a first tissue component with respect to a second tissue component, the method comprising joining the first and second tissue components with a tissue adhesive mixed with one or more compounds having free hydroxyl groups,
wherein the tissue adhesive comprises at least one prepolymer species comprising isocyanate-capped polymeric polyether-polyol and at least one species of polyisocyanate with molecular weight below about 2000 D, and
wherein the one or more compounds having free hydroxyl groups are mixed with the tissue adhesive at the time of its application to tissue.

2. The method of claim 1 wherein one or more compound having free hydroxyl groups is a polyether-polyol.

3. The method of claim 1 wherein one or more compound having free hydroxyl groups is selected to prevent swelling of the adhesive in water after it has cross linked.

4. The method of claim 1 wherein one or more compound having free hydroxyl groups is selected to prevent or diminish the formation of tissue adhesions.

5. The method of claim 1 wherein the first tissue component is a urethra.

6. The method of claim 1 where the first tissue component is herniated tissue.

7. The method of claim 1 wherein the first tissue component is herniated tissue selected from a rectocele, an enterocele, a cystocele, a cystoenterocele, an inguinal hernia, and a defect in the pelvic floor.

8. A method of supporting the urethra in a mammal to treat incontinence, the method comprising the steps of:
a) preparing a support assembly by applying, to a porous implantable sling material, a polymer comprising a polyol end-capped with at least one polyisocyanate group, and polymerizing the polymer to form a padded area on a porous implantable sling material;
b) passing the padded support assembly to the site at which the urethra is to be supported;
c) adjusting the location of the padded area of the assembly to a location at the point where the mesh places the urethra in tension; and
d) adjusting the tension on the assembly to provide support to the urethra.

9. The method of claim 8 wherein the mesh material is a polymerized isocyanate capped polyol.

10. The method of claim 8 wherein the mesh material is coated with a polymerized isocyanate capped polyol.

11. The method of claim 8 wherein at least one of the mesh, a coating on the mesh, and a pad on the mesh is constructed of a material that prevents local tissue adhesions.

12. A device for supporting a urethra, the device comprising a length of an implantable, porous, biocompatible mesh material shaped to be attached to an internal structure at the ends of the length of material, and constructed to have a padded region between the ends where the urethra can be supported without erosion by the support;
wherein the mesh material is one or both of a polymerized isocyanate-capped polyol, and a mesh material coated with a polymerized isocyanate-capped polyol.

13. The device of claim 12 wherein the mesh material is selected to be dimensionally stable, and the material for the padded area is selected to be resilient.

14. The device of claim 12 wherein at least one of the mesh, a coating on the mesh, and a pad on the mesh is constructed of a material that prevents local tissue adhesions.

15. A surgical method of repairing pelvic floor herniation comprising:
a) exposing the defective supporting tissue layer with an incision; b) optionally repairing the defect in the tissue layer with a first surgical adhesive; c) coating the exposed tissue layer with a second adhesive, which may be the same as or different from the first adhesive; d) applying force to return the herniated tissue to a proper anatomical position before the second adhesive cures; and e) closing the incision.

16. The method of claim 15 wherein the tissue layer is not repaired before applying the adhesive.

17. The method of claim 1 wherein the defective supporting tissue layer is one or more of the rectovaginal fascia, the rectus fascia, and the endopelvic fascia.

18. A surgical repair kit for herniation comprising one or more surgical adhesives, one or more delivery devices comprising applicator tips, and one or more of a mesh and a mesh-forming material;
wherein the surgical adhesive is a liquid composition comprising an isocyanate-capped polyether polyol and free polyisocyanate having molecular weight less than about 2000 D.

19. The kit of claim 18 further comprising at least one of an aqueous solution and a non-endcapped polyether polyol.

20. The kit of claim 18 wherein two surgical adhesives are included, one of which is immiscible with the second, and further including a mixing means that will temporarily create a suspended mixed state of the two adhesives that can then be loaded into the delivery means.

21. The kit of claim 18 wherein at least a portion of the polyol is bioabsorbable.

22. A method of stabilizing the pelvic floor, in which a continuous layer of surgical adhesive is applied with one surface of the layer in contact with the tissue to be reinforced and another surface in contact with one or more supporting structures selected from one or more of fascia, ligament, bone, and muscle, wherein the surgical adhesive is a liquid composition comprising an isocyanate-capped polyether polyol and free polyisocyanate having molecular weight less than about 2000 D.

23. The method of claim 22, in which the supporting structures are located so that when the surgical adhesive cures in the therapeutic position, the forces generated by the supporting structures are sufficient to maintain the therapeutic position of the tissue.

24. The method of claim 22 wherein the tissue to be reinforced is herniated tissue selected from a rectocele, an enterocele, a cystocele, a cystoenterocele, an inguinal hernia, and a defect in the pelvic floor.

25. The method of claim 22 in which the surgical adhesive bonds to one or more of the arcus tendinous fascia, the ileal pectineal muscle group, and the pubococcygeous muscles.

26. A method of treatment for rectocele, the method comprising:
making a transverse incision between the anterior rectal wall and posterior vaginal wall;
dissecting to separate the posterior wall of the vagina from the rectum;
injecting liquid adhesive into the floor of this space; and
pulling the posterior vaginal wall out and down to reapproximate the space; wherein polymerization of the adhesive simultaneously adheres the two planes and forms a malleable soft layer separating and supporting one or both of the rectum and the vagina.

27. The method of claim 26 wherein the tissue supported is the rectum.

28. The method of claim 26 wherein the space is dissected sufficiently to provide a place for a mesh.

29. The method of claim 26 wherein the malleable soft layer provides support without the use of sutures, staples or clips.

30. A method of using a surgical adhesive in pelvic floor reconstruction, the method including using a surgical adhesive to close ruptures of the tissues involved, and to close incisions made to repair the ruptures, wherein the adhesive is used in place of a mesh.

31. The method of claim 30 wherein use of the adhesive eliminates one or more incisions required when a mesh is used.

32. The method of claim 30 wherein the surgical adhesive is a liquid composition comprising an isocyanate-capped polyether polyol and free polyisocyanate having molecular weight less than about 2000 D.

33. The method of claim 30, wherein the pelvic floor reconstruction is done to treat one or more of direct or indirect inguinal hernia, cystocele, rectocele, enterocele, and cystoenterocele.

34. A method for forming an adherent supporting mesh on a site, the method comprising:
providing a first surgical adhesive component and a second surgical adhesive component, wherein the first and second components are not stably miscible, and wherein the first adhesive component degrades at the site more rapidly than the second component;
mixing the adhesive components;
applying the adhesive to a site and allowing the adhesives to separate into separate phases having a characteristic phase size significantly smaller than the size of the site;
and causing or arranging for the first and second adhesives to cure and to adhere to at least one surface at the site;
whereby the degradation of domains enriched with the first adhesive creates voids within a mesh formed by the second adhesive.

35. An adhesive composition, wherein the adhesive composition comprises at least one species of isocyanate-capped polymeric polyether-polyol, at least one species of polyisocyanate with molecular weight below about 2000 D, and one or more diluent compounds mixed with the adhesive composition at the time of administration, the diluent being one or more of polymeric polyols and water.

36. The composition of claim 35 wherein the diluent compound is a polymeric compound that is the same as, or is substantially similar in subunit composition to, the polyether-polyol used to make the isocyanate-capped polymeric polyether-polyol.

37. The composition of claim 35 wherein the polymeric polyol is selected to prevent substantial swelling of the adhesive, after it has cross linked, when exposed to aqueous solutions.

38. The composition of claim 35 wherein the compound with free hydroxyl groups comprises a polyether polyol present in sufficient quantity in the adhesive composition to prevent the adhesive, after it has cross linked, from swelling in aqueous solutions.

39. The composition of claim 35, further comprising a reinforcing material selected from a mesh and a dispersed fibrillar material.

40. The composition of claim 39, wherein the reinforcing material is a woven or non-woven mass or sheet of fibril, or a dispersed fibrillar material, wherein the fibril or fibrillar material is formed from the same material as the adhesive composition.

41. The composition of claim 39, wherein the reinforcing material is a woven or non-woven mass or sheet of fibrils, or a dispersed fibrillar material, formed from one or more materials, wherein at least one material is different from the material of the adhesive composition.

42. The composition of claim 35 wherein the polyether-polyol is a copolymer of ethylene oxide with one or more of propylene oxide and other alkylene oxides.

43. The composition of claim 42 wherein the proportion of alkylene oxide units in the polyether polyol that are not derived from ethylene oxide is no more than about 30% by number.

44. The composition of claim 35 wherein the adhesive composition comprises subunits that cause the polymerized adhesive composition to degrade in situ in the body in a reasonably predictable time.

45. The composition of claim 35 wherein the polymerized adhesive composition lacks subunits that would cause it, after polymerization in the body, to degrade in situ in the body in a reasonably predictable time.

46. The composition of claim 35 wherein the adhesive composition forms a partially permeable sheet after polymerization.

47. The composition of claim 46 wherein the permeability is formed by the phase separation, before or during polymerization, of species of isocyanate-capped polyether-polyol having limited mutual solubility.

48. The composition of claim 46 wherein the permeability is formed over time after polymerization of the adhesive.

49. The composition of claim 46 wherein the adhesive comprises a leachable filler.

50. The composition of claim 46 wherein the adhesive comprises a first and a second species of isocyanate-capped polyether-polyol, and wherein the first species biodegrades more rapidly than the second species.

51. The use of the composition of claim 35 for the fixation of a urethra to improve urinary continence.

52. The use of claim 51 in conjunction with the use of an ancillary support device.

53. The use of claim 52 wherein the device is selected from one or more of a sling, a pad, a tube, and a mesh.

54. The use of the composition of claim 35 for the repair of a herniation.

55. The use of claim 54 wherein the herniation is selected from a rectocele, an enterocele, a cystocele, a cystoenterocele, and a direct or indirect inguinal hernia.

56. The use of the composition of claim 35 to repair the pelvic floor.

57. The use of the composition of claim 35 as a coating for part of or all of a medical device or implant.

58. The use of claim 57 wherein the coating is applied so as to make one portion of the device reject adhesions and other side be inflammatory or adhesion producing.

59. The use of the composition of claim 38 to prepare a non-swelling medical implant.

60. The use of the composition of claim 38 to prepare a non-swelling coating on a medical implant.
